(19) United States
(12) Patent Application Publication
(10) Pub. No.: US 2005/0085432 A1
(43) Pub. Date: Apr. 21, 2005

(54) Methods of using conjugates of saccharides and acetamidino or guanidino compounds for treating bacterial infections

(76) Inventor: Aviva Lapidot, Rehovot (IL)

Correspondence Address: Martin Moynihan;Anthony Castorina, Suite 207, 2001 Jefferson Davis Highway, Arlington, VA, 22202, US

(21) Appl. No.: 10/498,978

(22) PCT Filed: Dec. 16, 2002
(86) PCT No.: PCT/IL02/01038

Related U.S. Application Data
(60) Provisional application No. 60/342,085, filed on Dec. 26, 2001.

(51) Int. Cl. A61K 31/715

(52) U.S. Cl. 514/42; 514/61

(57) ABSTRACT

A method of treating a bacterial infection in an individual is provided. The method is effected by administering to the individual a therapeutically effective amount of a pharamaceutical composition including an acetamidino- or guanidino- conjugated saccharide.

### FIELD AND BACKGROUND OF THE INVENTION

The present invention relates to a method of treating bacterial infections using conjugates of saccharides and acetamidino or guanidino compounds.

Antibiotic resistance is a growing problem encountered with all classes of antibiotics. One of the first groups of antibiotics to encounter the challenge of resistance was the aminoglycoside-aminocyclitol family. Aminoglycosides constitute a large group of biologically active bacterial secondary metabolites, which are used in the treatment of serious bacterial infections, such as tuberculosis and nosocomial infections.

Initially, resistance was restricted to bacterial modification of the antibiotic targets. For instance, all streptomycin-resistant M. tuberculosis strains carry point mutations leading to alterations in the ribosome, the site targeted by the antibiotic agent. As new aminoglycosides came into use, chemical modification mechanisms of resistance became more widespread. Unlike penicillin resistance where antibiotic hydrolysis is the mechanism of action, resistance to aminoglycosides is mediated by enzymes, which catalyze co-factor dependent modification of the hydroxy or amino groups of aminocyclitol residues.

Aminoglycoside-modifying enzymes are characterized by several levels of aminoglycoside inactivation: ATP-dependent O-phosphorylation by phosphotransferases (APH), ATP-dependent O-adenylation by nucleotidyltransferases (ANT) and acetyl CoA-dependent N-acetylation by acetyltransferases. Over 50 different enzymes found in most Gram-negative and Gram-positive bacterial pathogens have been identified as aminoglycoside modifiers [Shaw, K J. et al. (1993) Microbiol. Rev. 57:138-163], including a chimeric enzyme, which protects strains that carry it from almost all available aminoglycosides.

Thus, with growing bacterial resistance to antibiotics, the challenge at present is to generate highly potent antibacterial agents, which are effective at treating resistant strains and yet not toxic for use in humans.

Several approaches have been undertaken to uncover novel antibiotic agents or make presently employed antibiotic agents effective in treating resistant strains.

Aminoglycoside-Derivatives

Several aminoglycoside derivatives were designed and tested. The effectiveness of such novel aminoglycoside-derivatives is examined in terms of antibacterial potency, degree of resistance to inactivation by microbial enzymes and potential toxicity. An assessment of a number of compounds structurally related to gentamycin, sisomicin, fortimicin and kanamycin, revealed that none had overall properties superior to their parental compounds. In no case did a compound prove to be less toxic, and in many instances, the antibacterial potency of the newer agents was lower than that exhibited by the older aminoglycosides, while only a slight increase in resistance to inactivating enzymes was seen (reviewed in Price, K E. et al. (1986) Am. J. Med. 80:182-189).

Protein Kinase Inhibitors

Recent crystal structures of APHs, showed high similarity between APH (3′)-IIIa and protein kinases, which encouraged the use of protein kinase inhibitors as APH inhibitors [Daigle, D M. Et al. (1997) J. Biol. Chem. 272:24755-24758]. Indeed, various inhibitors of serine/threonine and tyrosine kinases (e.g., the isoquinoline sulfonamides and the flavanoids genistein and quercetin) showed mid μM-inhibition of the APH enzymes, however reversal of antibiotic resistance was not observed.

Aminoglycoside Modifications

Synthesis of aminoglycoside molecules which have antibiotic properties and are poor substrates for modifying enzymes has also been attempted. For example, tobramycin and dibekacin lack the 3′-hydroxyl group which is the site of APH(3′)-catalyzed phosphorylation of kanamycin class of aminoglycosides, and as such are competitive inhibitors of APH(3′) and potentially useful as antibiotic agents [McKay, G A. et al (1995) J. Biol. Chem. 270:24686-24692, Umezawa, S. et al. (1971) J. Antibiot. 24:274-275]. Unfortunately, tobramycin and dibekacin serve as substrates for other aminoglycoside kinases such as APH(2″), which are frequently found in Gram-positive organisms [Daigle, D M. et at. (1999) J. Biol. Chem. 6:99-110].

In another approach, several analogues of kanamycin and neamine lacking either the NH₂ group or the OH group in positions that are common sites for AAC modification, but remote to typical kinase targets hydrolysis, were synthesized [Roestamadji, J. et al. (1995) J. Am. Chem. Soc. 117:11060-11069]. Several of these compounds were very poor substrates for APH(3′)-Ia and APH(3′)-IIa, and exhibited antimicrobial activity in E. coli containing these enzymes. Although this approach is promising it is limited by the fact that most of these compounds were effectively phosphorylated by APH(3′)-IIIa.

While reducing the present invention to practice the present inventors have uncovered that compositions that include an acetamidino- or guanidino-conjugated saccharide are capable of relieving and curing bacterial infections.

Thus, the present invention provides novel antimicrobial agents and methods of using same for treating bacterial infections even when such infections are caused by previously resistant strains of bacteria.

### SUMMARY OF THE INVENTION

According to one aspect of the present invention there is provided a method of treating a bacterial infection in an individual, the method comprising administering to the individual a therapeutically effective amount of a pharmaceutical composition including an acetamidino- or guanidino-conjugated saccharide.

According to another aspect of the present invention there is provided an article of manufacture comprising packaging material and a pharmaceutical composition identified for treatment of a bacterial infection being contained within the packaging material, the pharmaceutical composition including, as an active ingredient, an acetamidino- or guanidino-conjugated saccharide and a pharmaceutically acceptable carrier.

According to further features in preferred embodiments of the invention described below the acetamidino- or guanidino-conjugated saccharide is of a formula:

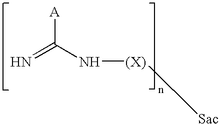

According to still further features in the described preferred embodiments A is CH₃ or NH₂; X is a linear or branched C₁-C₈ alkyl chain; n is an integer equal to or greater than 1; and Sac is the residue of a mono- or oligo-saccharide.

According to still further features in the described preferred embodiments n is an integer from 1 to 6.

According to still further features in the described preferred embodiments the alkyl chain includes a side group selected from the group consisting of a hydroxy group, an amino group and an oxo group.

According to still further features in the described preferred embodiments the acetamidino- or guanidino-conjugated saccharide is acetamidino-conjugated saccharide and whereas A is CH₃.

According to still further features in the described preferred embodiments the Sac is a monosaccharide.

According to still further features in the described preferred embodiments the active ingredient is methyl 6-deoxy-6-(N-acetamidino)-α-D-mannopyranoside.

According to still further features in the described preferred embodiments the Sac is an oligosaccharide.

According to still further features in the described preferred embodiments the oligosaccharide is a residue of an aminoglycoside antibiotic.

According to still further features in the described preferred embodiments the aminoglycoside antibiotic is selected from the group consisting of neomycin, kanamycin, sisomycin, fortimycin, paromomycin, neamine and gentamycin.

According to still further features in the described preferred embodiments the active ingredient is γ-(N-acetamidino) butyric acid-neomycin B.

According to still further features in the described preferred embodiments the active ingredient is tetra-γ-(N-acetamidino) butyric acid-kanamycin A.

According to still further features in the described preferred embodiments the active ingredient is guanidino-conjugated saccharide and whereas A is NH₂.

According to still further features in the described preferred embodiments the Sac is a monosaccharide.

According to still further features in the described preferred embodiments the acetamidino- or guanidino-conjugated saccharide is methyl 6-deoxy-6-guanidino-α-D-mannopyranoside.

According to still further features in the described preferred embodiments the active ingredient is methyl 6-deoxy-6-(N-L-argininamido)-α-D-mannopyranoside.

According to still further features in the described preferred embodiments the Sac is an oligosaccharide.

According to still further features in the described preferred embodiments the oligosaccharide is a residue of an aminoglycoside antibiotic.

According to still further features in the described preferred embodiments the aminoglycoside antibiotic is selected from the group consisting of neomycin, kanamycin, sisomycin, fortimycin, paromomycin, neamine and gentamycin.

According to still further features in the described preferred embodiments the acetamidino- or guanidino-conjugated saccharide is tetraargininamido-kanamycin A conjugate of a formula:

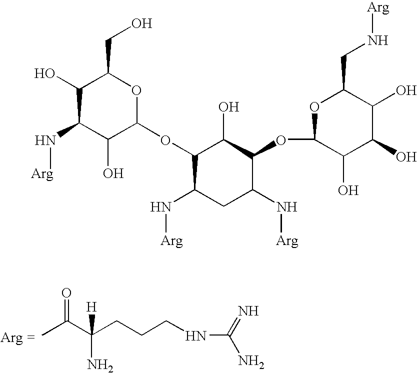

According to still further features in the described preferred embodiments the acetamidino- or guanidino-conjugated saccharide is triargininamido-gentamycin C conjugate of a formula:

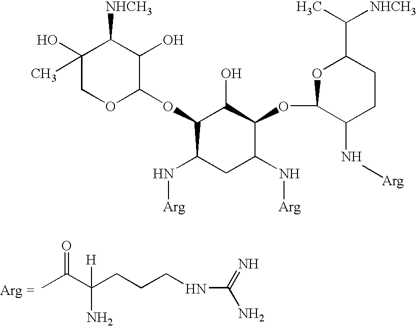

According to still further features in the described preferred embodiments the acetamidino- or guanidino-conjugated saccharide is tetraargininamido-gentamycin C conjugate of a formula:

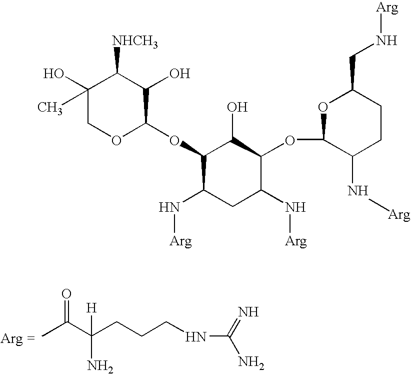

According to still further features in the described preferred embodiments the acetamidino- or guanidino- conjugated saccharide is hexa-argininamido-neomycin B conjugate of a formula:

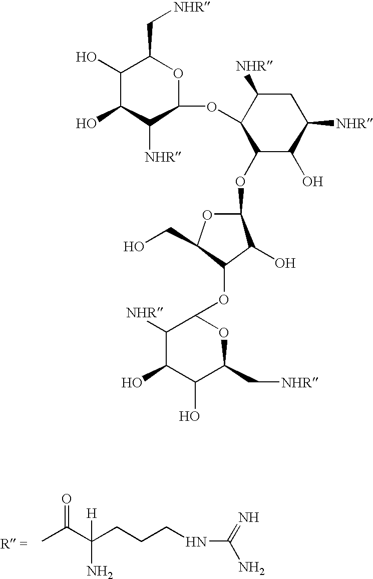

According to still further features in the described preferred embodiments the acetamidino- or guanidino-conjugated saccharide is tetraargininamido-neamine 1 conjugate of a formula:

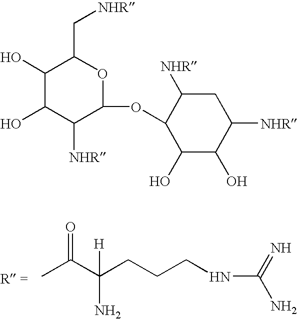

According to still further features in the described preferred embodiments the acetamidino- or guanidino-conjugated saccharide is a pentaargininamido-paramomycin conjugate of a formula:

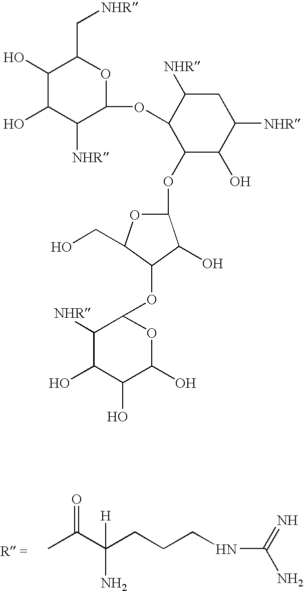

According to still further features in the described preferred embodiments the acetamidino- or guanidino-conjugated saccharide is γ-(N-guanidino)butyric acid-neomycin B conjugate of a formula:

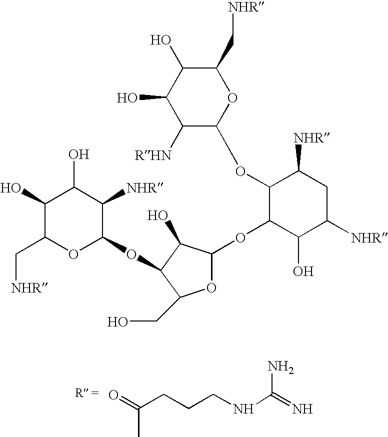

According to still further features in the described preferred embodiments the acetamidino- or guanidino-conjugated saccharide is a tetra-γ-(N-guanidino) butyric acid-kanamycin A conjugate of a formula:

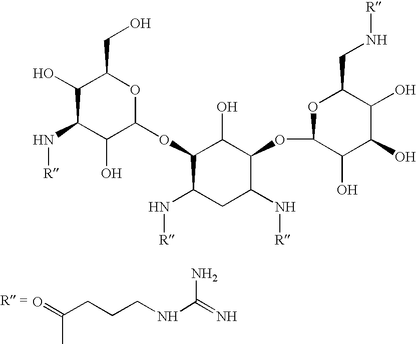

The present invention successfully addresses the shortcomings of the presently known configurations by providing a novel approach for treating bacterial infections using conjugates of saccharides and acetamidino or guanidino compounds.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 schematically illustrates the aminoglycoside-arginine conjugates utilized by the methods of the present invention.

FIG. 2 is a sequence alignment of a portion of the RNA-binding domain of RNase P retrieved from a number of bacterial strains. Grey boxes indicate an arginine-rich consensus sequence.

FIG. 3 is an autoradiogram depicting ptRNA processing mediated by RNase P of various bacterial strains, in the absence and presence of indicated concentrations of aminoglycoside-arginine conjugates.

FIGS. 4a-b illustrate ptRNA cleavage efficiency of E. coli RNase P as a function of increasing concentrations [nM] of NeoR (FIG. 4a) and R3G (FIG. 4b).

FIG. 5 is an autoradiogram depicting the effect of various concentrations of NeoR and R3G on ptRNA processing mediated by human RNase P.

FIG. 6 is an autoradiogram depicting the effect of indicated concentrations of polyA on the inhibition of E. coli RNase P activity by NeoR and R3G.

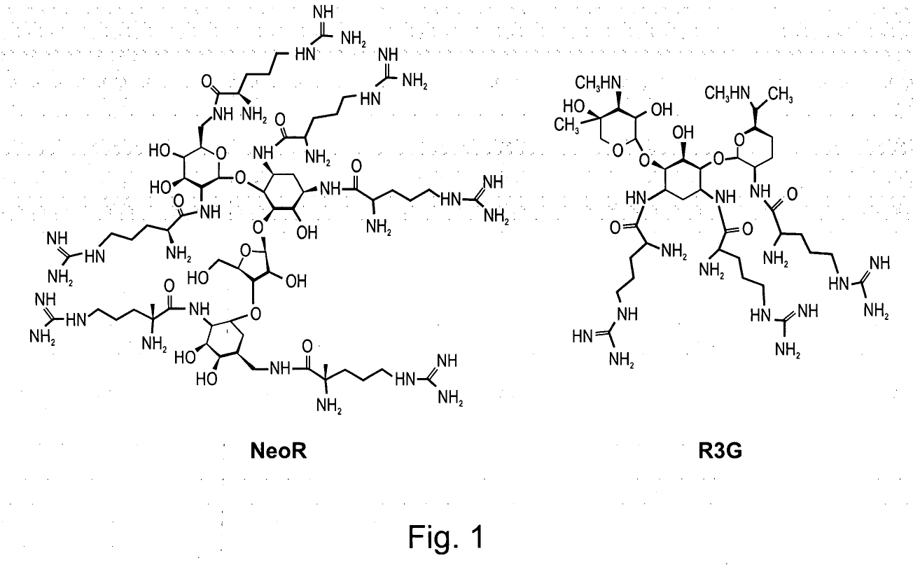

### DETAILED DESCRIPTION OF THE PREFERRED EMBODIMENTS

The present invention can be used for the treatment of bacterial infections. Specifically, the present invention employs conjugates of saccharides and acetamidino or guanidino compounds for the treatment of various bacterial diseases.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings described in the Examples section. The invention is capable of other embodiments or of being practiced or carried out in various ways. Also, it is to be understood that the phraseology and terminology employed herein is for the purpose of description and should not be regarded as limiting.

The aminoglycoside antibiotics are broad-spectrum antibacterial compounds that were used extensively for the treatment of many bacterial infections. However their increased use has led to the appearance of resistant bacterial strains. This, together with high cytotoxicity, limited the broad clinical use of such antibiotics.

While reducing the present invention to practice, the present inventors have uncovered that conjugates of saccharides and acetamidino or guanidino compounds, specifically, derivatives of aminoglycosides, are highly efficient as bacteriocidal/bacteriostatic agents.

As is further detailed hereinbelow, these conjugates enable treatment of bacterial infections even in cases where such infections are resistant to conventional antibiotic agents, or when toxicity of conventional antibiotics prevents utilization of an aggressive treatment regimen.

Although the complete mechanism of action of these conjugates is not thoroughly understood, it is conceivable that they interfere with bacterial targets i.e., RNA-protein complexes (RNP), thus blocking various biological processes necessary for pathogen growth and proliferation [for further details see the background of the Examples section and Eubank et al. (2002) FEBS Lett. 511:107-112].

A structural study of aminoglycoside-arginine conjugates (AACs) and HIV RNA target i.e., the trans-activator responsive element (TAR), enabled characterization of the structural determinants of aminoglycoside-arginine conjugates which are important for substrate recognition and affinity [Litovchick A. et al. (2001) Biochemistry, 40:15612-15623].

This study suggested that AAC binding is different than that of the parental aminoglycoside compounds. Binding of aminoglycoside-arginine conjugates to RNA targets is predicted to be a combination of specific binding of one arginine moiety with the bulge of TAR-RNA and non-specific interactions between the rest of the conjugate and the loop segment of TAR-RNA.

Thus, specific parameters that may contribute to binding affinity of the aminoglycoside conjugates are: (i) length and rigidity of the linker between the aminoglycoside core and the guanidine group of the arginine moiety; (ii) interaction between the α-amino of the aminoglycoside-arginine conjugate and the RNA target, as experimentally predicted by structural models of NeoR binding to TAR-RNA [Litovchick A. et al. (2000) Biochemistry 39:2838-2852]; (iii) multiple contact points gained from the interaction of at least one arginine and the bulge of TAR-RNA [Seewlad MJ. et al. (1998) J. Biomol. Struct. Dynamics 16:683-692 and Litovchick A. et al. (2000) Biochemistry 39:2838-2852].

Thus, according to one aspect of the present invention, there is provided a method of treating a bacterial infection in an individual. Preferred individual subjects according to the present invention are mammals such as canines, felines, ovines, porcines, equines, bovines, humans and the like.

The term “treating” refers to alleviating or diminishing a symptom associated with a bacterial infection. Preferably, treating cures, e.g., substantially eliminates, the symptoms associated with the infection and/or substantially decreases bacterial load in the infected tissue.

Bacterial infections treated according to the present invention include opportunistic aerobic gram-negative bacilli such as the genera Pseudomonas, bacterial infection caused by P. aeruginosa, bacterial infections caused by gram-positive bacilli such as that of the genus Mycobacterium, and mycobacteria, which causes tuberculosis-like diseases. A variety of bacterial infections may be treated by the method of the present invention, these include: M. tuberculosis, M. leprae, M. Intracellulare, M. smegmatis, M. bovis, M. kansasii, M. avium, M. scrofulcium, or M. africanum.

The method includes administering to the individual a therapeutically effective amount of an acetamidino- or guanidino-conjugated saccharide.

The saccharide according to the present invention may be a simple monosaccharide such as (i) pentose, e.g., arabinose, xylose, ribose and the like; (ii) disaccharide such as hexose, e.g., sucrose, maltose, lactose, cellobiose and the like; (iii) trisaccharide, e.g., mannotriose, raffmose, meleziose and the like; or (iv) a tetrasaccharide, e.g., amylopectin, Syalyl Lewis X (SiaLex) and the like. Alternatively, the saccharide can be a saccharide derivative such as, but not limited to, glucosides, ethers, esters, acids and amino saccharides.

A preferred saccharide of the present invention is a natural aminoglycoside antibiotic such as, but not limited, kanamycin, neomycin, seldomycin, tobramycin, kasugamycin, fortimicin, gentamycin, paromomycin, neamine and sisomicin. Alternatively, semi-synthetic derivatives of aminoglycosides such as amikacin, netilmicin and the like can also be used.

The saccharide residue may be linked to a spacer (X) through any suitable group, for example through an alkylene chain or, preferably, through an acylamino group.

The aminoglycoside-arginine conjugates (AACs) of the present are preferably of the following general formula: 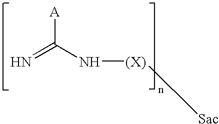
wherein A is NH₂ and X is (CH₂)₃—CH(NH₂)—C(═O)—.

Several conjugation schemes can be employed, including conjugation of one or more arginine derivative moieties to one or more saccharide cores. The conjugates preferably include short chains of L and D (n=1-6) arginines, although longer chains of n=10 or even n=20 are also envisaged. Alternative, conjugates can be α,ω-diamino acids of varying length such as β-alanine, ornithine and lysine (2,3 and 4 methylene groups, resepectively) or γ-amino acids such as glycine (aminoacetic acid), β-amino propionic acid or γ-amino butyric acid conjugated to aminoglycosides converted at the terminal amino groups into guanidine or N-acetamidino moieties. Examples of conjugates which can be utilized by the present invention include but are not limited to: 6-deoxy-6-(N-acetamidino)-α-D-mannopyranoside, γ-(N-acetamidino) butyric acid-neomycin B, tetra-γ-(N-acetamidino) butyric acid-kanamycin A, 6-deoxy-6-guanidino-α-D-mannopyranoside, 6-deoxy-6-(N-L-argininamido)-α-D-mannopyranoside, monoarginineamido-kanamycin A, monoarginineamido-gentamycin C, monoarginineamido-neomycin B, monoarginineamido-paramomycin, diarginineamido-kanamycin A, diarginineamido-gentamycin C, diarginineamido-neomycin B, diarginineamido-paramomycin, tetraargininamido-kanamycin A, triargininamido-gentamycin C, tetraargininamido-gentamycin C, hexa-argininamido-neomycin B, tetraargininamido-neamine 1, pentaargininamido-paramomycin, γ-(N-guanidino) butyric acid-neomycin B, tetra-γ-(N-guanidino) butyric acid-kanamycin A and the like [International Pat. NO: WO 00/39139, Litovchick et al. (1999) FEBS Lett. 445:73-79, Litovchick et al. (2000) Biochemistry 39:2838-2852 and Lapidot A. and Litovchick A. (2000) Drug Develop. Res. 50:502-515, Cabrera C. et al. (2000) AIDS Res. Hum. Retroviruses 16:627-634, Litovchick et al. (2001) Biochemistry 40:15612-15623, Cerebra et al. (2002) Antiviral research 53:1-8; Carriere et al. (2002) RNA 8:1267-1279 and Catani et al. (2002) J. Neurochemistry in-press].

The active ingredient (AAC) of the method of the present invention can be administered to an individual per se, or as part of a pharmaceutical composition where it is mixed with a pharmaceutically acceptable carrier.

As used herein a “pharmaceutical composition” refers to a composition of one or more of the active ingredients described hereinabove, or physiologically acceptable salts or prodrugs thereof, with other chemical components such as physiologically suitable carriers and excipients. The purpose of a pharmaceutical composition is to facilitate administration of a compound to an organism.

Hereinafter, the phrases “pharmaceutically acceptable carrier” and “physiologically acceptable carrier” are used interchangeably to refer to a carrier or a diluent that does not cause significant irritation to a treated individual and does not abrogate the biological activity and properties of the active ingredient.

Herein the term “excipient” refers to an inert substance added to a pharmaceutical composition to further facilitate administration of active ingredients. Examples, without limitation, of excipients include calcium carbonate, calcium phosphate, various sugars and types of starch, cellulose derivatives, gelatin, vegetable oils and polyethylene glycols.

Techniques for formulation and administration of the pharmaceutical compositions of the present invention may be found in “Remington's Pharmaceutical Sciences,” Mack Publishing Co., Easton, Pa., latest edition, which is incorporated herein by reference.

Suitable routes of administration may, for example, include oral, rectal, transmucosal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer a pharmaceutical composition in a local rather than systemic manner, for example, via injection of the composition directly into the area of infection often in a depot or slow release formulation, such as described below.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredient into compositions which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the pharmaceutical composition can be formulated by combining the active agents with pharmaceutically acceptable carriers well known in the art. Such carriers enable the pharmaceutical composition used by the method of the invention to be formulated as tablets, pills, dragees, capsules, liquids, gels, syrups, slurries, suspensions, and the like, for oral ingestion by a patient. Pharmacological compositions for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose compositions such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the agents for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the active ingredient and a suitable powder base such as lactose or starch.

Ophthalmic formulations, eye ointments, powders, solutions and the like, are also contemplated as being within the scope of this invention.

The compositions described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain fornulatory agents such as suspending, stabilizing and/or dispersing agents.

Pharmaceutical compositions for parenteral administration include aqueous solutions of the active ingredient in water-soluble form. Additionally, suspensions of the active ingredient may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or formulations, which increase the solubility of the active ingredient to allow for the composition of highly concentrated solutions.

Alternatively, the active ingredient may be in powder form for constitution with a suitable vehicle, e.g., sterile, pyrogen-,free water, before use.

The composition of the present invention may also be formulated in rectal compositions such as suppositories or retention enemas, using, e.g., conventional suppository bases such as cocoa butter or other glycerides.

In addition to the formulations described previously, a composition of the present invention may also be formulated for local administration, such as a depot composition. Such long acting formulations may be administered by implantation (for example subcutaneously or intramuscularly) or by intramuscular injection. Thus, for example, the composition may be formulated with suitable polymeric or hydrophobic materials (for example, as an emulsion in an acceptable oil) or ion exchange resins, or as sparingly soluble derivatives such as sparingly soluble salts. Formulations for topical administration may include, but are not limited to, lotions, suspensions, ointments gels, creams, drops, liquids, sprays emulsions and powders.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of active ingredient effective to prevent, alleviate or ameliorate symptoms of disease or prolong the survival of the subject being treated.

Determination of a therapeutically effective amount is well within the capability of those skilled in the art, especially in light of the detailed examples provided herein (see Example 1 of the Examples section).

For any composition used by the methods of the invention,. the therapeutically effective amount or dose can be estimated initially from cell culture assays and cell-free assays (See Example 2 and Example 3 of the Examples section). For example, a dose can be formulated in animal models to achieve a circulating concentration range that includes the IC₅₀ as determined in in-vitro assays. Such information can be used to more accurately determine useful doses in humans.

The AACs utilized by the present invention exhibit far greater affinity towards their cellular targets than their parental compositions (see Example 1 of the Examples section below), and as such, low concentrations/quantities thereof may be used in treatment of various bacterial infections, thereby avoiding cytotoxicity. In particular, cytotoxicity analysis showed that NeoR is not toxic to mice when administered as two single doses of 25 mg/kg body weight for the duration of two hours [Litovchick A. et al. (2001) Biochemistry40: 15612-15623].

Regardless, toxicity and therapeutic efficacy of the pharmaceutical compositions described herein can be determined by standard pharmaceutical procedures in experimental animals, e.g., by determining the IC₅₀ and the LD₅₀ (lethal dose causing death in 50% of the tested animals) for a subject ingredient. The data obtained from assays can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma levels of the active ingredient, which are sufficient to maintain the 21 required effects, termed the minimal effective concentration (MEC). The MEC will vary for each composition, but can be estimated from in vitro data; e.g., the concentration necessary to achieve 50-90% inhibition (see Example 1 of the Examples section). Dosages necessary to achieve the MEC will depend on individual characteristics and route of administration. HPLC assays or bioassays can be used to determine plasma concentrations.

Dosage intervals can also be determined using the MEC value. Compositions should be administered using a regimen, which maintains plasma levels above the MEC for 10-90% of the time, preferable between 30-90% and most preferably 50-90%.

It is noted that, in the case of local administration or selective uptake, the effective local concentration of the drug may not be related to plasma concentration. In such cases, other procedures known in the art can be employed to determine the effective local concentration.

Depending on the severity and responsiveness of the infection to be treated, dosing can also be a single administration of a slow release composition, with course of treatment lasting from several days to several weeks or until cure is effected or diminution of the infection state is achieved.

The amount of a composition to be administered will, of course, be dependent on the subject being treated, the severity of the infection, the manner of administration, the judgment of the prescribing physician, etc.

Compositions of the present invention can be packaged in a dispenser device, as one or more unit dosage forms as part of an FDA approved kit, which preferably includes instruction for use, dosages and counter indications. The kit can include, for example, metal or plastic foil, such as a blister pack suitable for containing pills or tablets, or a dispenser device suitable for use as an inhaler. The kit may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising an active ingredient suitable for use with the present invention may also be prepared, placed in an appropriate container, and labeled for treatment of an indicated disease or condition.

Many diseases and conditions associated with bacterial infections are difficult if not impossible to treat using commercially available antibiotics due to bacterial resistance and drug-associated cytotoxicity.

The bacteriocidal activity of acetoamido- or guanido-saccharide conjugates makes such compounds highly suitable for treating bacterial infections even in cases where prolonged treatment regimens are necessary. As such, these compounds may play a pivotal role in the fields of therapy and antibiotic design in years to come. Furthermore, the incomparable affinity and specificity that the conjugates of the present invention have towards bacterial RNA (see Example 2 of the Examples section) may serve as a basis for the development of a diagnostic assay for premature detection of bacterial infections. The proposed novel assay may be far more specific and reliable than present methods.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes 1-111 Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### BACKGROUND

RNase P is a ubiquitously expressed enzyme, which catalyzes processing of the 5′ termini of precursor tRNAs (ptRNAs) and other cellular RNAs (e.g., p4.5S RNA) which are involved in protein biosynthesis [xiao, S. et al. (2001) J. Cell Physiol. 187:11-21, Altman, S. (1999) “The RNA World” Cold Spring Harbor Laboratory Press, Cold Sping Harbor , N.Y. 2^{nd} edition 351-380 and Harris, M E. Et al. (1998) “RNA Structure and Function” Cold Spring Harbor Laboratory Press, Cold Spring Harbor , N.Y. 309-337]. The bacterial RNase P holoenzyme is composed of a catalytic RNA moiety (˜350-400 nucleotides) and a protein co-factor (˜110-150 amino acid residues).

RNase P recognizes the ptRNA structure via interactions between the catalytic RNA subunit and the T- and acceptor-stems mainly, although residues in the 5′-leader sequence as well as the 3′-terminal sequence also contribute to such interactions. The protein subunit of RNase P apparently also affects substrate recognition as well as the range of substrates, which can be used by RNase P. Although the RNA subunit can catalyze the ptRNA processing reaction in-vitro under non-physiological conditions [Guerrier-Takada, C. et al. (1983) Cell 35:849-857], probably due to its role in substrate recognition, the protein subunit is vital for RNase P activity in-vivo [Schedl, P. et al. (1973) Proc. Natl. Acad. Sci. 70:2091-2095 and Kurz, J C. et al. (2000) Curr. Opin. Chem. Biol. 4:553-558].

Thus, inhibition of bacterial RNase P activity is a major goal for drug designers due to its essential role in bacterial protein synthesis. In addition, due to its unique structure, which is not shared with the human enzyme, the bacterial holoenzyme, represents an excellent drug target.

### EXAMPLE 1

### Inhibition of In-Vitro Reconstituted Bacterial RNase P Activity by Aminoglycoside-Aginine Conjugates

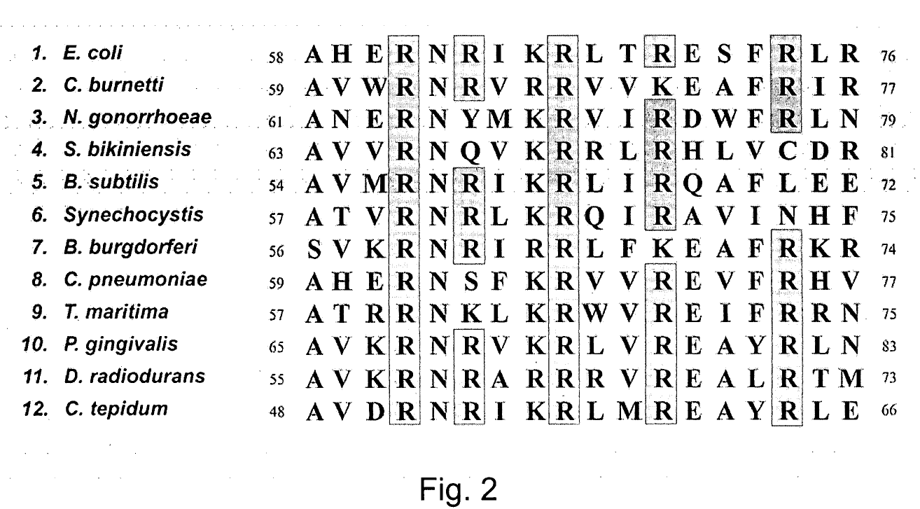

The ability of Aminoglycosides-arginine conjugates (AACs) to inhibit bacterial RNase P was investigated due to observations that (i) aminoglycosides interact with the RNA subunit of E. coli RNase P in vitro and interfere with its ptRNA-processing activity [Mikkelsen, N E. et al. (1999) Proc. Natl. Acad. Sci. 96:6155-6160] and (ii) sequence analysis of the protein subunit of RNase P from various bacterial species revealed an arginine-rich consensus, encompassed in the RNA-binding domain (RNR motif) of the RNase P protein co-factor [see FIG. 2, Vioque, A. et al. (1988) J. Mol. Biol. 202:835-848 and Gopalan, V. (1997) J. Mol. Biol. 267:818-829].

Materials and Methods

Reagents

Oligonucleotides for PCR were synthesized at HHMI Biopolymer/Keck Foundation Resource laboratory, Yale university School of medicine, New Haven Conn. Restriction and modifying enzymes were obtained from New England Biolabs, Beverlt Mass. and Gibco Life Technologies, Rockville, Md. T7 RNA ploymerase and Rnasin were purchased from Promega, Madison, Wis. Hi Trap columns and γ-[³²P]-GTP were obtained from Amersham Pharmacia Biotech. All other reagents used were purchased from Sigma-Aldrich St. Louis, Mo. and Fisher Biotech, Pittsburgh, Pa.

RNA, Protein and Inhibitor Preparation Synthesis and Purification

Polynucleotide sequences of Neisseria gonnorhoeae, Porphyromonas gingivalis and Streptococcus pneumoniae (SEQ ID NOS: 1, 3 and 5, respectively) expressing amino acid (SEQ ID NOS: 2, 4 and 6, respectively) subunits of RNase P were PCR amplified using standard PCR methodology. The genes encoding the RNA subunit of RNase P were cloned into pUC19 under the transcriptional control of a T7 RNA polymerase promoter. T7 RNA polymerase-mediated run-off in vitro transcription was performed on individual clones to generate the respective RNase P RNAs, which were then purified using Quick Spin columns. cDNAs encoding the protein subunits of the various bacterial species were subcloned into either expression vectors: pCRT7TOPO or pBAD (Invitrogen, Carlsbad, Calif.). Proteins were over-expressed in E. coli as His₆-tagged fusion proteins and purified to homogeneity using a combination of cation exchange and immobilized metal affinity chromatography.

DNA sequences were confirmed by DNA sequencing and molecular weight of the respective proteins was determined by electrospray ionization mass spectrometry.

RNase P from E. coli was prepared and purified according to Vioque, A. et al. (1988) J. Mol. Biol. 202:835-848 and Gopalan, V. (1997) J. Mol. Biol. 267:818-829.

Synthesis of NeoR and R3G was described elsewhere [Litovchick, A. et al. (1999) FEBS Lett. 445:73-79, Litovchick, A. et al. (2000) Biochemistry 39:2838-2852, Lapidot, A. et al. (2000) Drug Develop. Res. 50:502-515 and Litovchick, A. et al. Biochemistry in press].

ptRNA^{Tyr}su3+ was prepared by in vitro transcription of FokI-digested pUC19TyrT [Vioque, A. et al. (1988) J. Mol. Biol. 202:835-848].

RNase P Activity Assay

RNase P activity was determined in the presence or absence of AAC inhibitors suspended in 50 mM Tris-Hcl (7.2), 5 % (w/v) polyethylene glycol 8000, 1 mM NH₄Cl, 10 mM spermidine, 10 mM MgCl₂. Reactions were carried under multiple-tumover conditions (for example, 100 nM of radio-labeled ptRNA^{Tyr}su3+ and 0.5 nM E. coli RNase P holoenzyme).

Following holoenzyme assembly, AAC inhibitors were added to the reaction mixture and incubated for 5 minutes prior to the addition of [³²P]-ptRNA^{Tyr}su3+ substrate. Reactions were allowed to proceed for the indicated times and were terminated by adding a quenching dye [7 M Urea, 10 mM EDTA, 10% (v/v) phenol]. Reaction products were resolved by gel electrophoresis (8% polyacrylamide/7 M Urea) and auto-radiograms were obtained.

Extent of substrate cleavage was quantified using a PhosphorImager (Molecular Dynamics) and ImageQuant softwares. Initial cleavage velocity was calculated only from those reactions exhibiting substrate cleavage lower than 30%.

Results

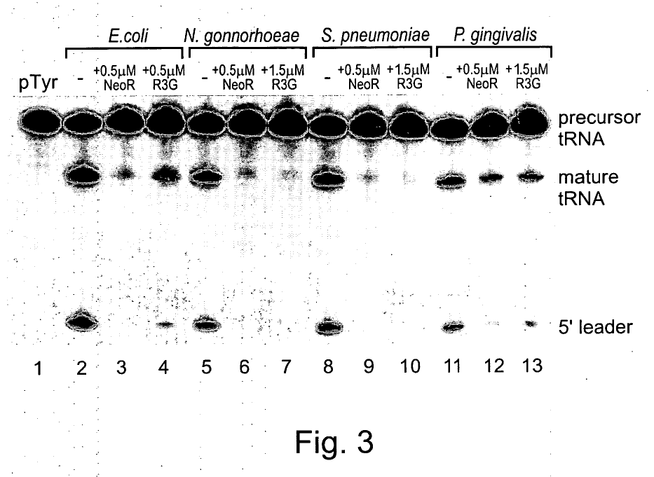

Reconstituted RNase P activity was tested in the presence or absence of indicated concentrations of AAC inhibitors. As shown in FIG. 3, in the absence of AAC inhibitors, radiolabeled-ptRNA^{Tyr}su3+ was well processed by E. coli RNase P and in particular by enzymes derived from N. gonnorhoeae and S. pneumoniae; less effective was ptRNA processing mediated by P. gingivalis. Addition of AAC inhibitors, either 500 nM NeoR or 1500 nM R3G to the reaction mixture resulted in nearly complete inhibition of RNase P processing activity; RNase P activity derived from P. gingivalis was less susceptible to the addition of the indicated inhibitors.

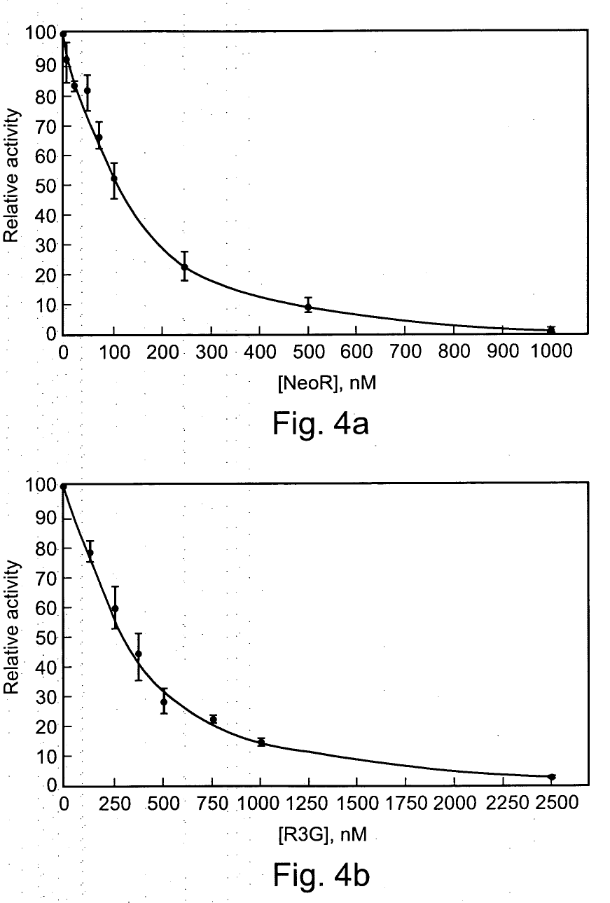

IC₅₀ values (i.e., concentration of inhibitor required to reduce enzymatic activity by 50% as observed in the absence of inhibitor) of NeoR and R3G were determined in the presence of increasing concentrations of either inhibitors. Initial reaction velocities were determined at various concentrations of each inhibitor. As shown in FIG. 4a-b, NeoR (FIG. 4a) and R3G (FIG. 4b) inhibited E. coli RNase P activity with IC₅₀ values of 125 nM and 300 nM, respectively. Further results suggest that IC₅₀ values for NeoR and R3G-mediated inhibition of various bacterial RNase P are in the sub-micromolar concentration range (FIG. 4). The IC₅₀ value of NeoR is 100-fold lower than that presented by the parental aminoglycoside [FIG. 4, Mikkelsen, Nebr. et al. (1999) Proc. Natl. Acad. Sci. 96:6155-6160].

### EXAMPLE 2

### Specificity of Aminoglycoside-Arginine Conjugates Towards Prokaryotic RNase P

RNase P functions as an RNP complex in all living organisms, however considerable variation in composition and structure exist. Compared to the simple composition and structure of bacterial RNase P (e.g., one RNA subunit: one protein subunit), the human holoenzyme is characterized by a higher level of complexity [Xiao, S. et al. (2001) J. Cell Physiol. 187:11-21]. In addition to a 340-nucleotide long RNA subunit, at least eight protein subunits ranging in size from 14 kDa to 115 kDa are found in association with the RNA subunit of human RNase P. Interestingly, none of the protein subunits posses the conserved arginine-rich tract found in bacterial RNase P. Moreover, the eukaryotic RNA subunit of RNase P is catalytically inactive in-vitro unlike its bacterial counterpart.

In order to determine if the AAC inhibitors of the present invention cross-react with human RNase P, the activity of a partially purified human enzyme was tested in the absence or presence of various concentrations of NeoR and R3G.

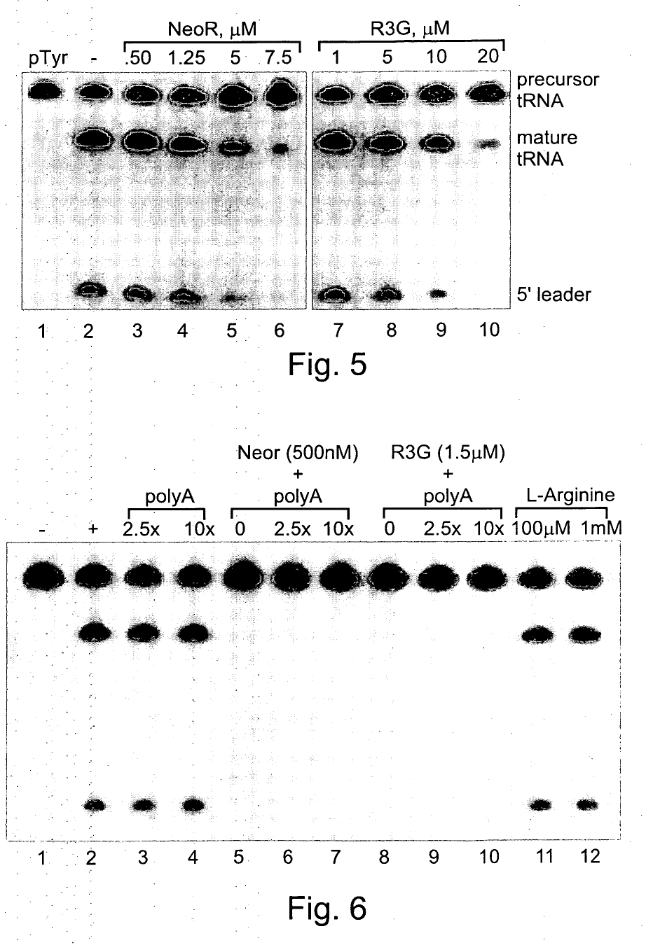

Results are shown in FIG. 5. Although human RNase P activity was largely unaffected at concentrations, which were 10-fold greater than the IC₅₀ values of NeoR and R3G for E. coli RNase P, a nearly complete inhibition of the human enzyme was observed at NeoR and R3G concentrations of 7.5 μM.

From these results it can be construed that the AACs utilized by the present invention are more effective in inhibiting bacterial RNase P than human RNase P.

### EXAMPLE 3

### Specificity of Aminoglycoside-Arginine Conjugates Towards RNase P

Positively charged compounds may serve as general inhibitors of any negatively charged biological molecule and as such of RNA. In order to determine whether the aminoglycoside-arginine conjugates of the present invention are specific inhibitors of RNase P, the inhibitory effect of NeoR and R3G on E. coli RNase P was examined in the presence or absence of various concentrations of positively charged molecules.

As shown in FIG. 6,addition of an 18-mer polyA oligonucleotide (lanes 24) or L-Arginine (lanes 10-11) did not inhibit RNase P specific activity even at 10-fold excess concentration over that of the ptRNA substrate used in the assay. Furthermore, addition of as much as 1 μM poly A RNA, failed to alter the ability of NeoR or R3G to inhibit E. coli RNase P (lanes 5-10).

These results are consistent with the finding that the inhibitory potential of NeoR and R3G vary dependent on the source of enzyme (see, FIG. 3 and FIG. 5), and with the reported observation that a 10-fold excess of tRNA had no effect on the ability of R3G to disrupt the RNP complex formed between HIV TAR RNA and Tat-derived peptide [Litovchick A. (2001) Biochemistry submitted for publication], again indicating that aminoglycoside-arginine conjugates have only a weak affinity to tRNAs.

Therefore it may be concluded that the inhibition of bacterial RNase P by NeoR and R3G is not due to their ability to bind non-specifically the ptRNA substrate and thereby interfere with RNase P catalysis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

1. A method of treating a bacterial infection in an individual, the method comprising administering to the individual a therapeutically effective amount of a pharamaceutical composition including, as an active ingredient, an acetamidino- or guanidino-conjugated saccharide.

2. The method of claim 1, wherein said acetamidino- or guanidino-conjugated saccharide is of a formula: 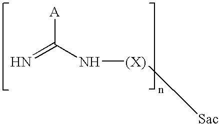
wherein A is CH₃ or NH₂; X is a linear or branched C₁-C₈ alkyl chain; n is an integer equal to, or greater than 1; and Sac is the residue of a mono- or oligo-saccharide.

3. The method of claim 2, wherein n is an integer from 1 to 6.

4. The method of claim 2, wherein said alkyl chain includes a side group selected from the group consisting of a hydroxy group, an amino group and an oxo group.

5. The method of claim 2, wherein said acetamidino- or guanidino-conjugated saccharide is acetamidino-conjugated saccharide and whereas A is CH₃

6. The method of claim 5, wherein Sac is a monosaccharide.

7. The method of claim 6, wherein said acetamidino- or guanidino-conjugated saccharide is methyl 6-deoxy-6-(N-acetamidino)-α-D-mannopyranoside.

8. The method of claim 5, wherein Sac is an oligosaccharide.

9. The method of claim 8, wherein said oligosaccharide is a residue of an aminoglycoside antibiotic.

10. The method of claim 9, wherein said aminoglycoside antibiotic is selected from the group consisting of neomycin, kanamycin, sisomycin, fortimycin, paromomycin, neamine and gentamycin.

11. The method of claim 10, wherein said acetamidino- or guanidino-conjugated saccharide is γ-(N-acetamidino) butyric acid-neomycin B.

12. The method of claim 10, wherein said acetamidino- or guanidino-conjugated saccharide is tetra-γ-(N-acetamidino) butyric acid-kanamycin A.

13. The method of claim 2, wherein said acetamidino- or guanidino-conjugated saccharide is guanidino-conjugated saccharide and whereas A is NH₂.

14. The method of claim 13, wherein Sac is a monosaccharide.

15. The method of claim 14, wherein said acetamidino- or guanidino-conjugated saccharide is methyl 6-deoxy-6-guanidino-α-D-mannopyranoside.

16. The method of claim 14, wherein said acetamidino- or guanidino-conjugated saccharide is methyl 6-deoxy-6-(N-L-argininamido)-α-D-mannopyranoside.

17. The method of claim 14, wherein Sac is an oligosaccharide.

18. The method of claim 17, wherein said oligosaccharide is a residue of an aminoglycoside antibiotic.

19. The method of claim 18, wherein said aminoglycoside antibiotic is selected from the group consisting of neomycin, kanamycin, sisomycin, fortimycin, paromomycin, neamine and gentamycin.

20. The method of claim 19, wherein said acetamidino- or guanidino-conjugated saccharide is tetraargininamido-kanamycin A conjugate of a formula:

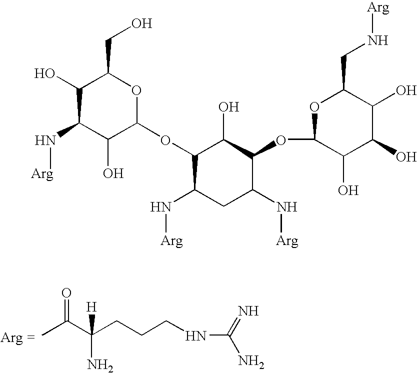

21. The method of claim 19, wherein said acetamidino- or guanidino-conjugated saccharide is triargininamido-gentamycin C conjugate of a formula:

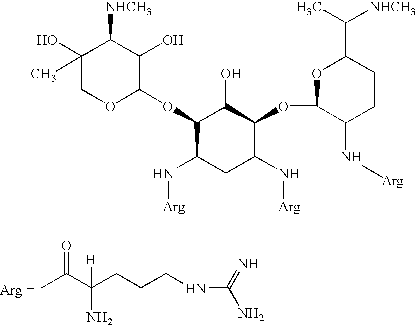

22. The method of claim 19, wherein said acetamidino- or guanidino-conguated saccharide is tetraargininamido-gentamycin C conjugate of a formula:

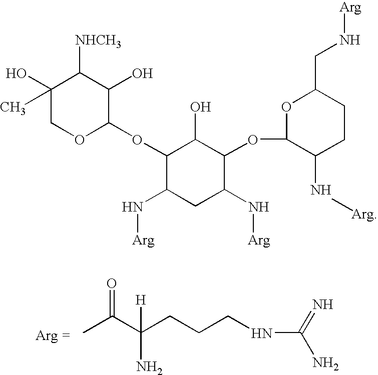

23. The method of 19, wherein said acetamidino- or guanidino-conguated saccharide is hexa-argininamido-neomycin B conjugate of a formula:

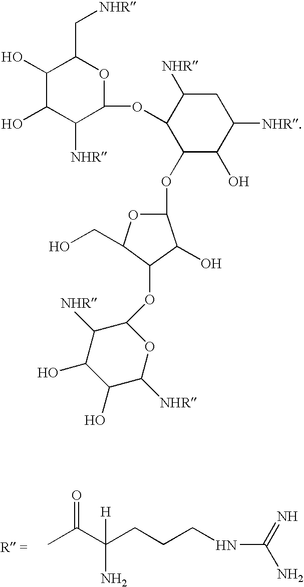

24. The method of claim 19, wherein said acetamidino- or guanidino-conjugated saccharide is tetraargininamido-neamine 1 conjugate of a formula:

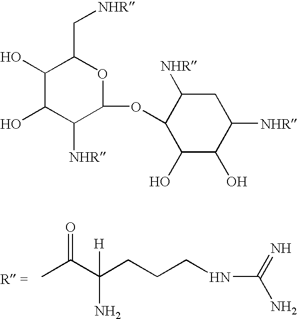

25. The method of claim 19, wherein said acetamidino- or guanidino-conjugated saccharide is pentaargininamido-paramomycin conjugate of a formula:

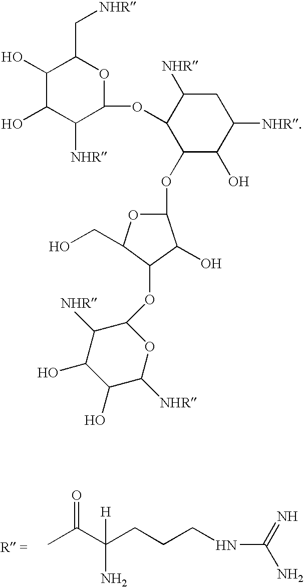

26. The method of claim 19, wherein said acetamidino- or guanidino-conjugated saccharide is γ-(N-guanidino) butyric acid-neomycin B conjugate of a formula:

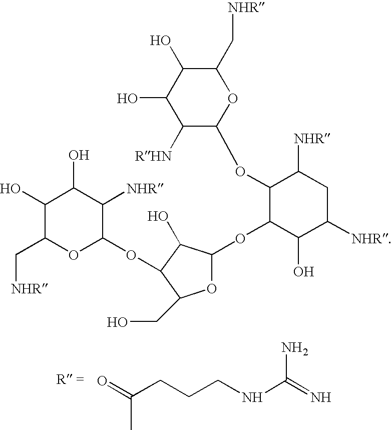

27. The method of claim 19, wherein said acetamidino- or guanidino-conjugated saccharide is tetra-γ-(N-guanidino) butyric acid-kanamycin A conjugate of a formula:

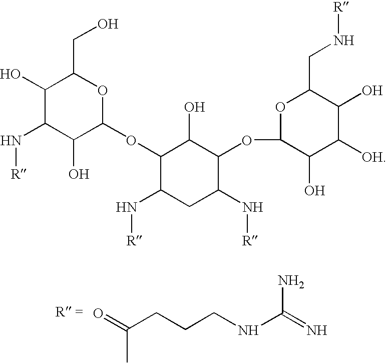

28. An article of manufacture comprising packaging material and a pharmaceutical composition identified for treatment of a bacterial infection being contained within said packaging material, said pharmaceutical composition including, as an active ingredient, an acetamidino- or guanidino-conjugated saccharide and a pharmaceutically acceptable carrier.

29. The article of manufacture of claim 28, wherein said acetamidino- or guanidino-conjugated saccharide is of a formula: 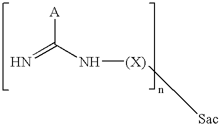
wherein A is CH₃ or NH₂; X is a linear or branched C₁-C₈ alkyl chain; n is an integer equal to, or greater than 1; and Sac is the residue of a mono- or oligo-saccharide.

30. The method of claim 29, wherein n is an integer from 1 to 6.

31. The article of manufacture of manufacture of claim 29, wherein said alkyl chain includes a side group selected from the group consisting of a hydroxy group, an amino group and an oxo group.

32. The article of manufacture of claim 29, wherein acetamidino- or guanidino-conjugated saccharide ingredient is acetamidino-conjugated saccharide and whereas A is CH₃.

33. The article of manufacture of claim 32, wherein Sac is a monosaccharide.

34. The article of manufacture of claim 32, wherein said acetamidino- or guanidino-conjugated saccharide is methyl 6-deoxy-6-(N-acetamidino)-α-D-mannopyranoside.

35. The article of manufacture of claim 32, wherein Sac is an oligosaccharide.

36. The article of manufacture of claim 35, wherein said oligosaccharide is a residue of an aminoglycoside antibiotic.

37. The article of manufacture of claim 36, wherein said aminoglycoside antibiotic is selected from the group consisting of neomycin, kanamycin, sisomycin, fortimycin, paromomycin, neamine and gentamycin.

38. The article of manufacture of claim 37, wherein said acetamidino- or guanidino-conjugated saccharide is γ-(N-acetamidino) butyric acid-neomycin B.

39. The article of manufacture of claim 37, wherein said acetamidino- or guanidino-conjugated saccharide is tetra-γ-(N-acetamidino) butyric acid-kanamycin A.

40. The article of manufacture of claim 29, wherein said acetamidino- or guanidino-conjugated saccharide is guanidino-conjugated saccharide and whereas A is NH₂.

41. The article of manufacture of claim 40, wherein Sac is a monosaccharide.

42. The article of manufacture of claim 41, wherein said acetamidino- or guanidino-conjugated saccharide is methyl 6-deoxy-6-guanidino-α-D-mannopyranoside.

43. The article of manufacture of claim 41, wherein said acetamidino- or guanidino-conjugated saccharide is methyl 6-deoxy-6-(N-L-argininamido)-α-D-mannopyranoside.

44. The article of manufacture of claim 30, wherein Sac is an oligosaccharide.

45. The article of manufacture of claim 44, wherein said oligosaccharide is a residue of an aminoglycoside antibiotic.

46. The article of manufacture of claim 45, wherein said aminoglycoside antibiotic is selected from the group consisting of neomycin, kanamycin, sisomycin, fortimycin, paromomycin, neamine and gentamycin.

47. The article of manufacture of claim 46, wherein said acetamidino- or guanidino-conjugated saccharide is tetraargininamido-kanamycin A conjugate of a formula:

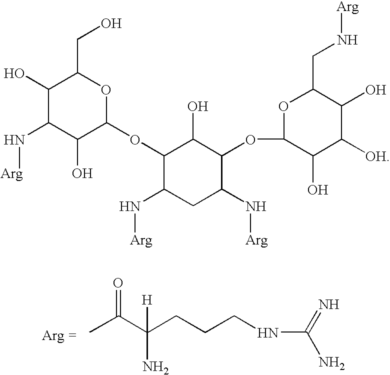

48. The article of manufacture of claim 46, wherein said acetamidino- or guanidino-conjugated saccharide is triargininamido-gentamycin C conjugate of a formula:

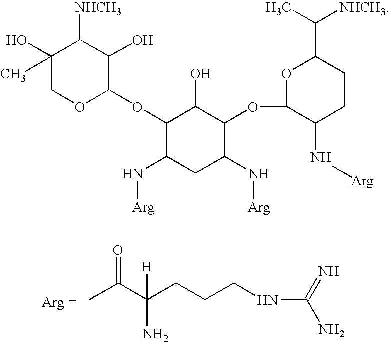

49. The article of manufacture of claim 46, wherein said acetamidino- or guanidino-conjugated saccharide is tetraargininamido-gentamycin C conjugate of a formula:

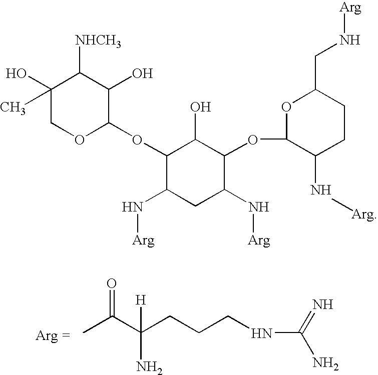

50. The article of manufacture of claim 46, wherein said acetamidino- or guanidino-conjugated saccharide is hexa-argininamido-neomycin B conjugate of a formula:

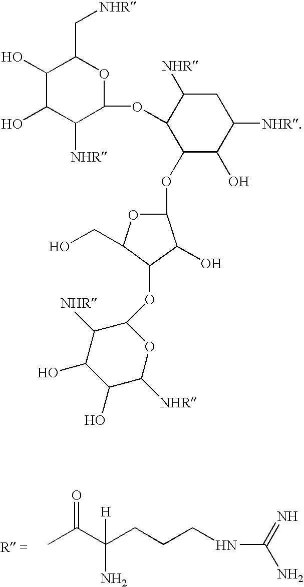

51. The method of claim 46, wherein said acetamidino- or guanidino-conjugated saccharide is tetraargininamido-neamine 1 conjugate of a formula:

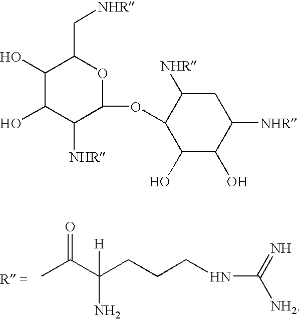

52. The method of claim 46, wherein said acetamidino- or guanidino-conjugated saccharide is pentaargininamido-paramomycin conjugate of a formula:

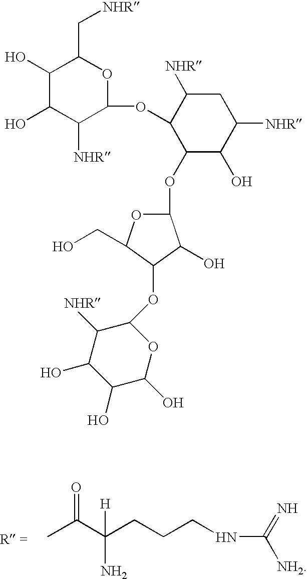

53. The article of manufacture of claim 46, wherein said acetamidino- or guanidino-conjugated saccharide is γ-(N-guanidino) butyric acid-neomycin B conjugate of a formula:

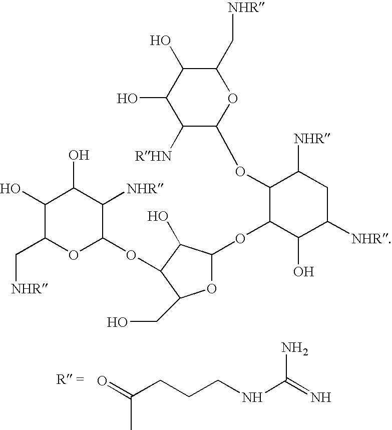

54. The article of manufacture of claim 46, wherein said acetamidino- or guanidino-conjugated saccharide is tetra-γ-(N-guanidino) butyric acid-kanamycin A conjugate of a formula: [chemical structure]


SEQUENCE LISTING (6 sequences)

SEQ ID NO: 1 - RNA, 354 nt, Neisseria gonorrhoeae
cgggacgggcagacagucgccgcguaucgcguaaggcauacggggaggaaaguccgggcuccgcaggguagaaugccgguuaacggccgggcgcgguaacgcgacggaaaguggaacagaaagcaaaaccgccgauggcugcuuuggcagcacaggcaagggugaaaaggugcgguaagggcgcaccgcgcauuugguaacaauaugcggcaggccaaaccccauucggagcaagaccaaacagaacgcaaugacgcugcccgccgagcguucggguagguugcuugagcauaccggcaacgguaugccuagaggaaugacuguccgagacagaacccggcuuaccgccugucc

SEQ ID NO: 2 - protein, 123 aa, Neisseria gonorrhoeae
MILDYRFGRQYRLLKTDDFSSVFAFRNRRSRDLLQVSRSNGNGLDHPRIGLVVGKKTAKRANERNYMKRVIRDWFRLNKNRLPPQDFVVRVRRKFDRATAKQARAELAQLMFGNPATGCGKQV

SEQ ID NO: 3 - RNA, 392 nt, Porphyromonas gingivalis
cagcagaucggucugucgcucacucuuucgagagugggaggaacguccgggcaacgcagagcaccauccuuccuaacaggaaguuacucgugaggguaaaggagcguagaagagaaugaccgccauuuuccaugaguugucugugcuucgguacggacuacguggaggguaagggugagaaggugggguaagagccuaccggaugcagcggugacgcugcaugccguacgucugauggguuguaagaucauguauaccggcgcauguaggguggcucgcccgaugccgggggguagaucgcuggagccaugcggugacgcacggccaagauaaaugacagacgcucuggcuacguguggccgugaguacagaacccggcuuacagaucugcu

SEQ ID NO: 4 - protein, 137 aa, Porphyromonas gingivalis
MTSPPTFGLSKSERLYLRDEINTVFGEGKAFVVYPLRVVYRLGSEHRVAYSSMLVSVAKKRFRRAVKRNRVKRLVREAYRLNKHLLNDVLQERQIYATIAFMVVSDELPDFRTVERAMQKSLIRIAGNVPSSALKNE

SEQ ID NO: 5 - RNA, 400 nt, Streptococcus pneumoniae
augugcaauuuuuggauaaucgcgugaggagaauuguuucucaugaggaaaguccaugcuagcacaggcugugaugccuguaguguuugugcuaggcgaaaccauaagccuagggacgagaaaucguuacggcaguugaaauggcuaaguccuuggauaggccagaguaggcuugaaagugccacagugacggagucuuucuggaaacagagagaguggaacgcgguaaaccccucaagcuagcaacccaaauuuuggucggggcauggaguacgcggaaacgaacguaguauucugacugcuaucagcuagagcuguuagugguagacagaugauuaucgaaggaagugguccuagucacuucuggaacaaaacauggcuuauagaaaauugcauauag

SEQ ID NO: 6 - protein, 124 aa, Streptococcus pneumoniae
MLKKNFRVKREKDFKAIFKEGTSFANRKFVVYQLENQKNRFRVGLSVSKKLGNAVTRNQIKRRIRHIIQNAKGSLVEDVDFVVIARKGVETLGYAEMEKNLLHVLKLSKIYREGNGSEKETKVD